(19) United States
(12) Patent Application Publication
(10) Pub. No.: US 2015/0136873 A1
(43) Pub. Date: May 21, 2015

(54) Scenting Device with Synchronized Audio Playback and On Demand Scenting

(71) Applicants: Scott Holmes Stapleford, Londonderry, NH (US); Spencer Levy, Miami, FL (US); Gary Nelson Stapleford, Londonderry, NH (US)

(72) Inventors: Scott Holmes Stapleford, Londonderry, NH (US); Spencer Levy, Miami, FL (US); Gary Nelson Stapleford, Londonderry, NH (US)

(21) Appl. No.: 14/086,505

(22) Filed: Nov. 21, 2013

(51) Int. Cl. A61L 9/12

(52) U.S. Cl. CPC A61L 9/122
USPC 239/69

(57) ABSTRACT

A system that synchronizes a disbursed scent with a related audio playback message in a retail or public environment. This system is controlled and managed off-site via the Internet with a specially designed software application.

### FIELD OF THE INVENTION

Embodiments of this invention generally relate to controlling a scenting device via a controller that synchronizes a disbursed scent with a related audio playback message. Control of the scenting devices can be performed via a hardwired or wireless connection. Scenting and audio playback are scheduled through the controller hardware via an Internet connection or can be triggered on-demand via a wireless remote.

### BACKGROUND

Scenting in a retail and public environment is common practice today and is accomplished using several different methods which include but are not limited to oils, gels and liquids that are either sprayed or evaporated into the an by a fan. The trigger to disperse these scents is predominantly done based upon an interval on-off timer or running a fan continuously over the scent media with no real logic behind when these actions occur.

Current scenting methods do not allow for a “call to action” with any sort of audio queue when the scent is disbursed and noticed by the customer in the retail or public environment—the scent is just simply disbursed with hopes that the target audience will know what to do. Additionally current systems do not allow for rewrote controlled “on-demand” scenting by the retailer when the target audience is near the product display.

Many methods today exist for adding scenting in a retail or public environment but the lack of an audio message in conjunction with the scent and on-demand scenting does not exist. What is needed in the art is a complete scenting & audio system that synchronizes both of these “human” senses together in a cohesive and easy-to-use delivery system that generates a “call to action” to the target audience of the scenting and audio system.

### SUMMARY

Systems according to some embodiments of this invention provide control hardware which is installed on-site in the retail or public environment. This control hardware is connected to the Internet for communication to the off-site control computer, the on-site public address system and the scenting hardware. Connection from the control hardware to the scenting hardware is done by hardwire connection or wireless communication.

In some embodiments, the control hardware which is connected to the Internet communicates with an off-site computer running specially designed control software. The control software allows remote management of these off-site units via a GUI (Graphical User Interface). The control software allows control of certain features of the remote units which include but are not limited to location, IP connection information, audio messages, volume levels, playlists, schedules and scenting synchronization.

In some embodiments, when the scenting and audio system is installed and has connectivity to the Internet, the retailer would program on-site control units with the control software. In the control software the retailer would program a scent to be disbursed when an audio message is played on the environments public address system. The retailer could pick aspects of how the scent is disbursed by pre-scenting before the message is played to make sure there is good coverage of the scent, scent for a specific amount of time during and after the scent is disbursed and select the channel to scent (if there are multiple scents to disburse).

In some embodiments, a wireless remote control for on-site “on-demand” scenting is achieved with a specially designed RF remote that is coded to transmit and activate scenting units which have built-in RF receivers.

### BRIEF DESCRIPTION OF THE DRAWINGS

FIG. 1 is a block diagram of the scenting system.

FIG. 2 is a front and rear perspective view of the scenting unit.

FIG. 3 is a rear perspective view of scenting unit and scent pack insertion.

FIG. 4 is an internal perspective view of the scenting unit.

FIG. 5 is a front and rear perspective view of the control unit.

FIG. 6 is a screen view of the control software.

### DETAILED DESCRIPTION

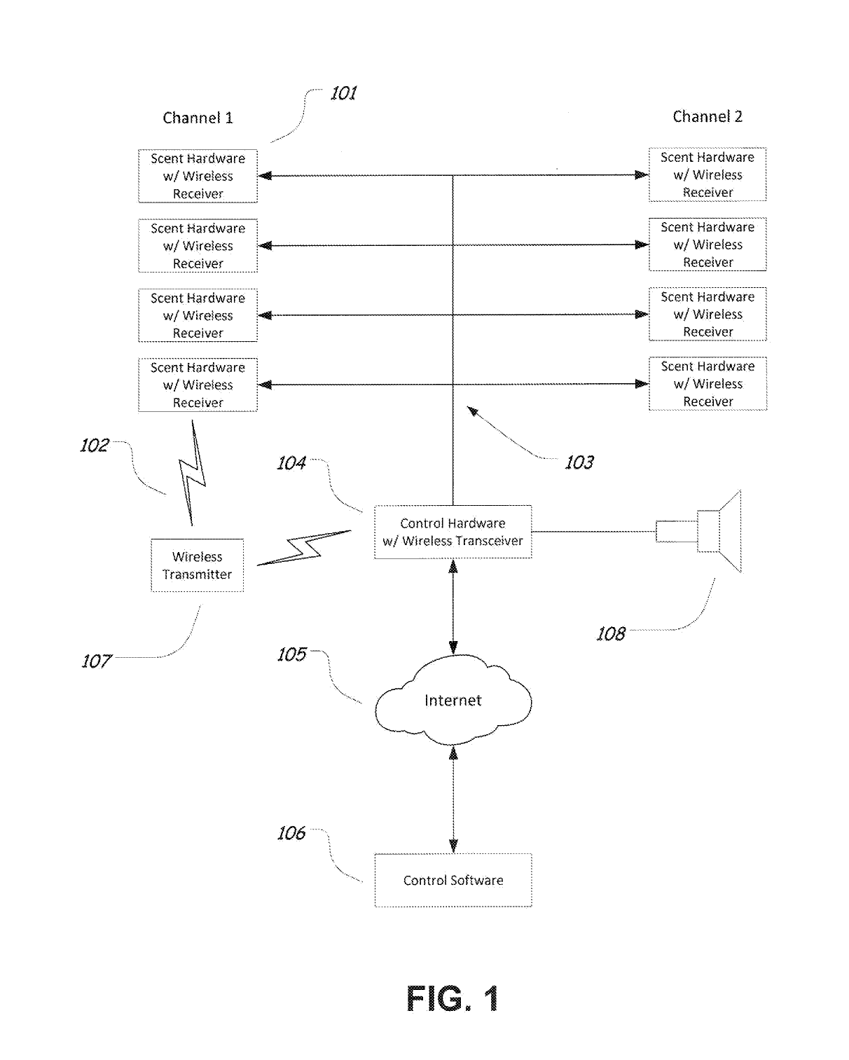

FIG. 1 illustrates one example of many possible ways to interconnect the system in a retail or public environment. The control hardware 104 would connect to the on-site Internet connection 105, public address system 108 & scenting hardware 101. The scenting hardware 101 could be connected to the control unit 104 by hardwire connection 103 or wireless communication 102. Off-site control of the control hardware 104 would be managed by the control software 106 running on a pc connected to the Internet 105. Local “on-demand” triggering of the scent hardware 101 could also be done through a wireless transmitter 107 via RE communication 102.

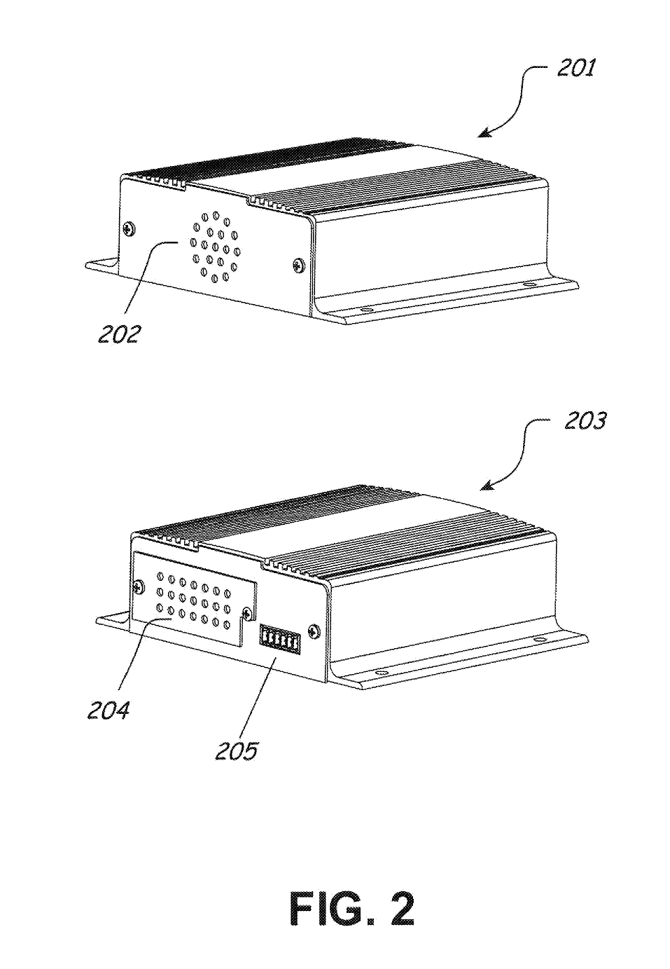

Certain embodiments of the invention are comprised of a scenting hardware unit as shown in FIG. 2 for disbursement of the scent media. The scenting hardware front view 201 consists of an opening 202 for a scenting air outtake. The scenting hardware rear view 203 consists of a door 204 that allows for an air intake and a control connector 205 that allows for hardwired control, daisy chaining of other scent hardware and power.

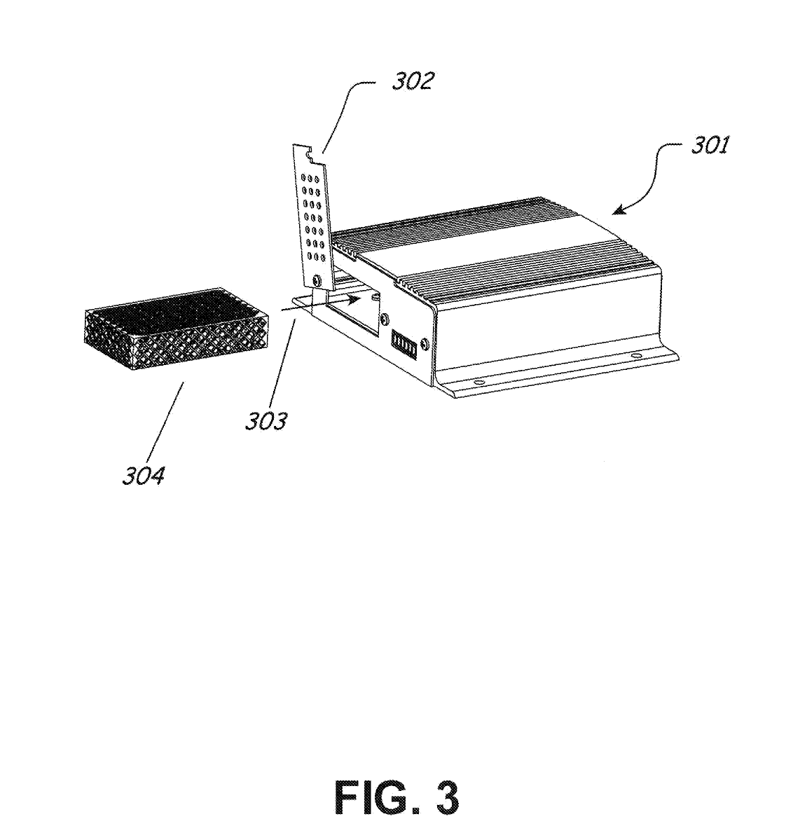

Certain embodiments of the invention are comprised of a scenting media pack that is inserted into the rear of the scenting hardware as shown in FIG. 3. The scenting hardware 301 would have a swing-up door 302 that would allow the insertion 303 of the scent media pack 304.

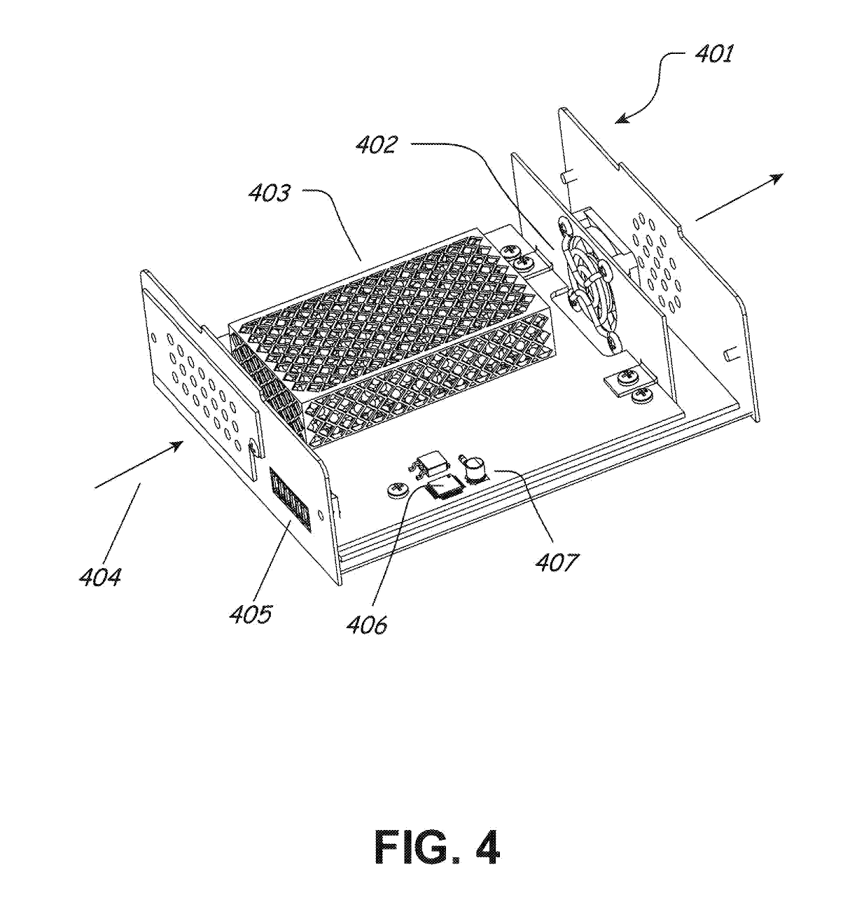

Certain embodiments of the invention are comprised of a scenting hardware unit with an electronically controlled fan and wireless receiver as shown in FIG. 4, The scenting hardware 401 would be controlled through the control connector 405 or wireless receiver 406. When a control signal is sent to the scent hardware 401 the control electronics 407 would turn the fan 402 on or off. When the fan 402 is on it would create air flow 404 through the scent media pack 403 and out the front of the scent hardware 401.

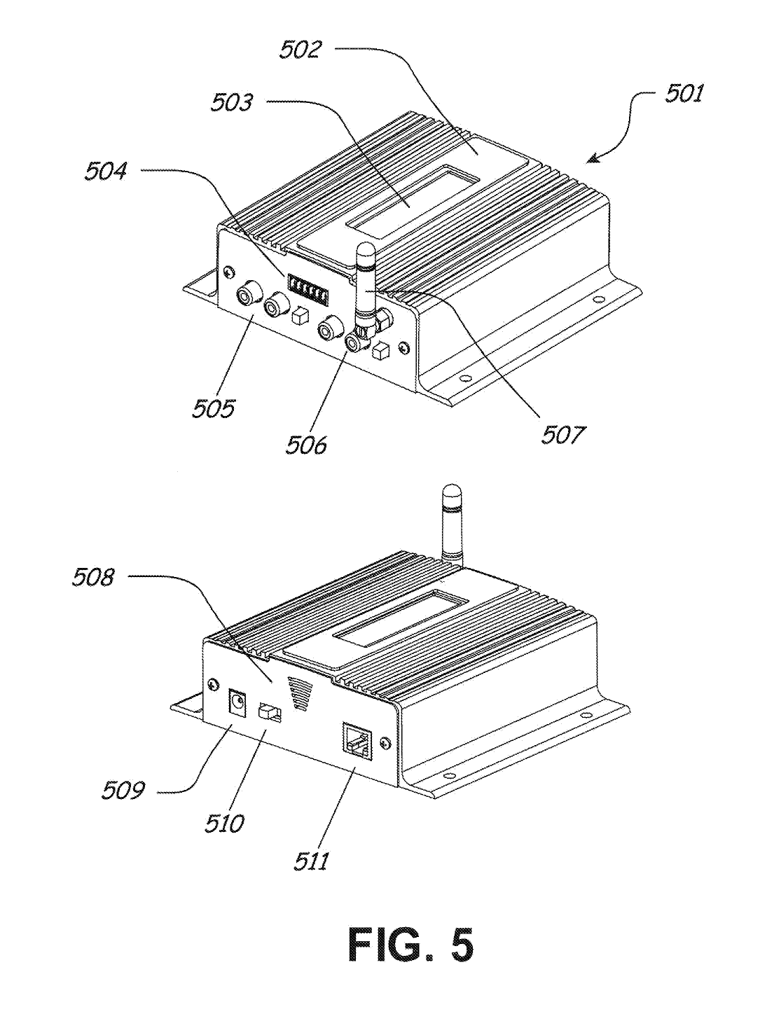

Certain embodiments of the invention are comprised of a control unit as shown in FIG. 5. The control unit 501 consists of a membrane keypad 502 and LCD display 503 for local user interaction. RCA connections 505 & 506 would connect to the locations public address system and wireless communications would be received and transmitted through the antenna 507. Power is supplied through the DC jack 509 with switching on and off done though the power switch 510. The Internet is connected through the RJ-45 jack 511. The control hardware's 501 audio output can be monitored through the built-in speaker through the speaker grill 508.

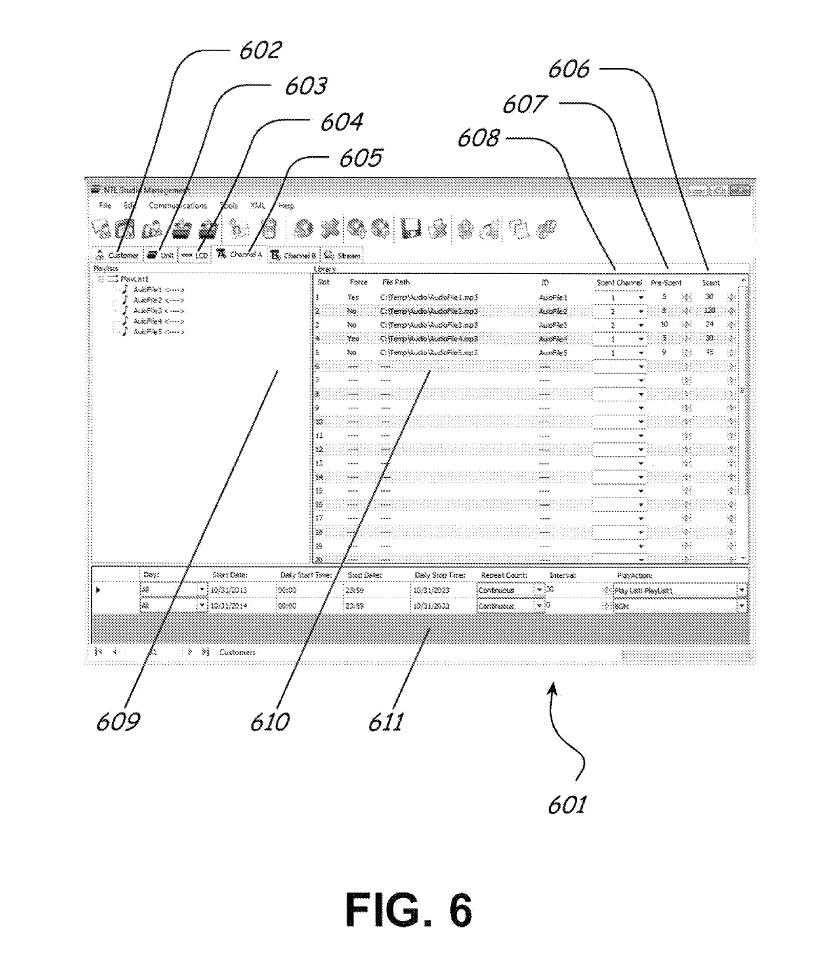

Certain embodiments of the invention comprise of control software that are used to connect to the control hardware via the Internet as shown in FIG. 6. The control software 601 allows the programming of on-site control hardware as shown in FIG. 5. The control software 601 consists of a GUI (Graphical User Interface) with tabs that allow different programming features which include, customer information 602, unit IP information 603, and LCD information 604. Audio and scent programming is created on the channel tabs 605. The channel tabs consist of a library area 610 where audio files added and can be bound to a scenting action. This scenting action can be programmed to scent a specific output zone 608, pre-scent before the audio message 607 and scent during and after the message 606 for a specific amount of time. Each line item action in the library 610 then can be placed in playlists 609 and then scheduled 611 for specific times and dates.

What is claimed is:

1. A system for disbursement of scent with an audio message comprising:
a. A control unit and scenting unit that synchronizes the scent with an auditory message.
b. A control unit that is Internet connected for remote management.

2. The system of claim 1, further comprising of wireless receiver for remote “on-demand” triggering of the system.

3. The system of claim 1, where the system has multiple scenting output channels.

4. The system of claim 1, that allows programmable pre-scenting.
